(19) United States
(12) Patent Application Publication
(10) Pub. No.: US 2019/0216569 A1
(43) Pub. Date: Jul. 18, 2019

(54) METHOD AND APPARATUS FOR GUIDING A SURGICAL INSTRUMENT TO A TARGET LOCATION

(71) Applicants: Modher Ibrahim Al-Shawi, Bulleen (AU); Ibrahim Mudhir Ahmed Al-Shawi, Amman (JO)

(72) Inventors: Modher Ibrahim Al-Shawi, Bulleen (AU); Ibrahim Mudhir Ahmed Al-Shawi, Amman (JO)

(21) Appl. No.: 16/246,551

(22) Filed: Jan. 14, 2019

Related U.S. Application Data
(60) Provisional application No. 62/617,367, filed on Jan. 15, 2018.

(51) Int. Cl. A61B 90/11; A61B 17/34

(52) U.S. Cl. CPC A61B 90/11; A61B 2090/571; A61B 2017/3405; A61B 17/3403

(57) ABSTRACT

An apparatus comprising a pivot mechanism configured to be mounted to a supporting structure, an arcuate arm operatively engaged to the pivot mechanism and a needle guide mounted to the arcuate arm. The pivot mechanism is pivotable about a pivot axis passing through a center of spherical rotation of the apparatus, the arcuate arm is rotatable along a circular path centered around the center of spherical rotation and the needle guide is configured to guide a surgical instrument to move perpendicular to the circular path along a radial axis. Once a target location on a patient's body is set and the pivotal axis is fixed, the rotation around pivotal axis, sliding along the circular path and movement of the surgical instrument along of the radial axis enable a target location to be reached from a range of different locations and angles on a patient's body.

### CROSS-REFERENCE TO RELATED APPLICATIONS

This application claims the benefit of U.S. Provisional Application No. 62/617,367, filed Jan. 15, 2018, the contents of which are incorporated herein by reference.

### FIELD OF THE INVENTION

The present invention generally relates to medical devices. More specifically, the present invention relates to apparatus and method for guiding a surgical instrument to a target organ under limited available visual information.

### BACKGROUND OF THE INVENTION

During surgical interventions, there are numerous instances where a surgeon or an operator needs to target a specific location in the body of a patient with a needle, a catheter or other interventional means while guided only by two-dimensional imaging. One such case is Percutaneous nephrolithotomy (PCNL) procedure which is a commonly used procedure for management of renal calculus disease or renal stone removal. Establishing a good access to the desired location in the targeted kidney is the first and probably the most crucial step of this procedure. The procedure is normally carried out under the guidance of two dimensional fluoroscopy.

To assist in preoperative planning of access, proper radiological imaging is essential. Historically, Intravenous urography (IVU) was the preferred imaging method of most endourologists before PCNL. With the widespread availability of multiphase computed tomography (CT) scanners, allowing imaging during delayed phases of contrast excretion and the capacity for coronal reconstruction, computed tomography (CT) is now commonly used in the evaluation before PCNL. With preoperative imaging and planning, the surgeon can select the desired entry point and tract. However, carrying out this procedure during the operation is still a difficult step that requires considerable skill.

In some countries, urologists establish their own percutaneous renal access, but in other countries, the procedure is often performed by interventional radiologists. This crucial step has a very steep learning curve in a fluoroscopy guided access as it involves visualizing a three dimensional anatomy on a two dimensional fluoroscopy screen. At least one study has found that as few as 11% of urologists who perform PCNL achieve successful access to the desired location. This low success rate is attributed to probably a lack of skill. This is probably due to the difficulty in visualizing and mentally imbibing the three dimensional anatomy of the pelvicalyceal system on the two dimensional fluoroscopy screen.

Thus, a need exists for a method and an apparatus which can address the above-mentioned problems.

### OBJECTS OF THE INVENTION

An object of the present invention is to provide an apparatus for guiding a surgical instrument to a target location under diminished visual field and/or with use of a two-dimensional visual display only.

Another object of the present invention is to reduce error in insertion and targeting of surgical instruments in percutaneous procedures.

Yet another object of the present invention is to provide a method for maintaining spatial orientation of a surgical instrument to accurately target an organ in percutaneous procedures.

Still another object of the present invention is to provide an apparatus for guiding a surgical instrument which gives freedom to an operator or surgeon in selection of entry point and tract as per preference for performing a percutaneous procedure.

These as well as other objects of the present invention are apparent upon inspection of this specification, including the drawings and appendices attached hereto.

### SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present invention provides an apparatus for guiding a surgical instrument to a target organ in a percutaneous procedure. The apparatus comprises an arcuate arm, a pivot mechanism and a needle guide. The arcuate arm is arc-shaped and it is a fraction of a circle's circumference that the arc makes up. The arcuate arm is positioned over a patient by securing it to a support structure through the pivot mechanism. The pivot mechanism allows the arcuate arm to rotate around its axis. The needle guide disposed on the arcuate arm allows a mounted needle or any surgical instrument to move along a radial axis. The pivotal axis and the longitudinal axis of the surgical instrument mounted on the needle guide intersect at a single spherical rotation center which lies at the center of curvature of the arcuate arm. The slider assembly enables sliding of the arcuate arm around the center of rotation. Thus, once the target for accessing a known organ location by the surgical instrument mounted on the apparatus is set and the pivotal axis is fixed, it gives the operator of the apparatus the freedom to manipulate the apparatus in any spatial orientation for positioning the surgical instrument at a desired entry point and angle to access the target organ.

### BRIEF DESCRIPTION OF THE DRAWINGS

In order to describe the manner in which features and other aspects of the present disclosure can be obtained, a more particular description of certain subject matter will be rendered by reference to specific embodiments which are illustrated in the appended drawings. Understanding that these drawings depict only typical embodiments and are not therefore to be considered to be limiting in scope, nor drawn to scale for all embodiments, various embodiments will be described and explained with additional specificity and details through the use of the accompanying drawings in which:

FIG. 1 is a pictorial view of the surgical instrument guiding apparatus in accordance with an embodiment of the present invention;

FIG. 2 is an exploded view of the apparatus of FIG. 1;

FIG. 3A illustrates an embodiment of a slider assembly;

FIG. 3B illustrates an exploded view of the slider assembly of FIG. 3A;

FIG. 4A to FIG. 4D illustrate different embodiment of combinations of arcuate arm and slider assembly;

FIG. 5A to FIG. 5C illustrate different designs of an arcuate arm used in the apparatus of the present invention;

FIG. 6A illustrates a needle guide holder integral to the arcuate arm;

FIG. 6B illustrates a needle guide holder attachable to the arcuate arm;

FIG. 7A and FIG. 7B illustrate insertion of a needle guide into a needle guide holder;

FIG. 8A to FIG. 8F illustrate details of a needle holder disposed on the needle guide and a needle insertion process in accordance with an embodiment of the present invention;

FIG. 9A to FIG. 9D illustrate details of a needle holder disposed on the needle guide and a needle insertion process in accordance with another embodiment of the present invention;

FIG. 10 is a pictorial depiction of a patient, in the prone position, on an operating table undergoing a percutaneous surgery with the use of the surgical instrument guiding apparatus of the present invention; and

FIG. 11 is an illustration of the spherical coordinate reference frame used to calculate the desired position of the surgical instrument guiding apparatus of the present invention for carrying out an interventional procedure on a patient.

### DETAILED DESCRIPTION OF THE INVENTION

Embodiments of the present invention are described herein in the context of an apparatus and method for providing a guide for PCNL entry. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

A spherical mechanism is a rotational manipulator with all axes intersecting at the center of the sphere. The present invention uses a spherical mechanism to guide a needle or a catheter or other surgical interventional means to a desired location in the body of a patient.

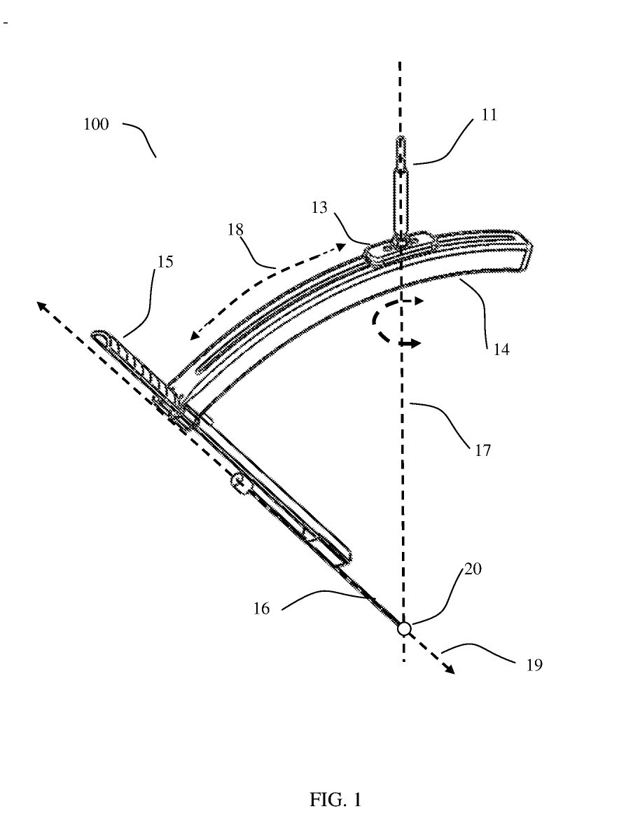

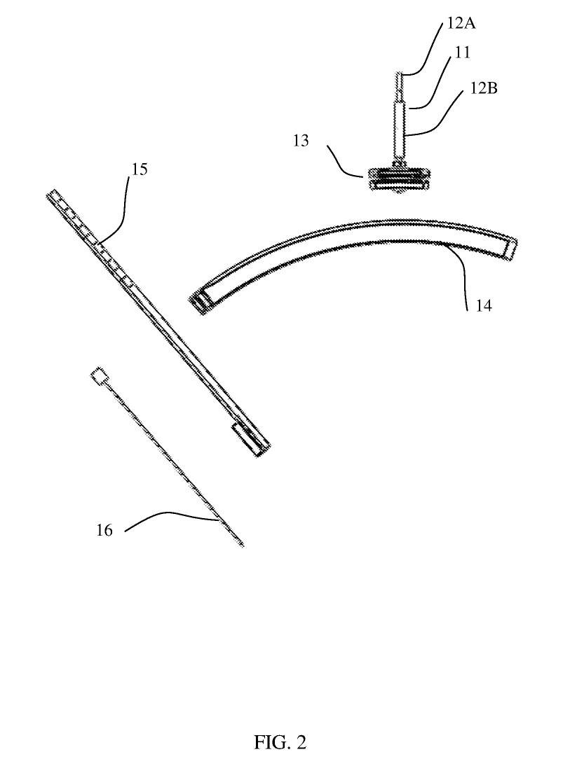

The surgical instrument guiding apparatus 100 of the present invention, as shown in FIG. 1 and FIG. 2, comprises a pivot mechanism 11, an arcuate arm 14 and a needle guide 15.

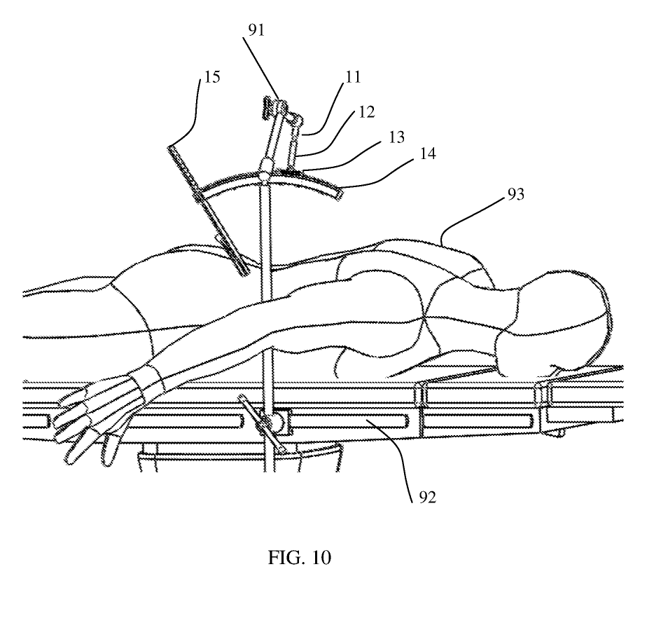

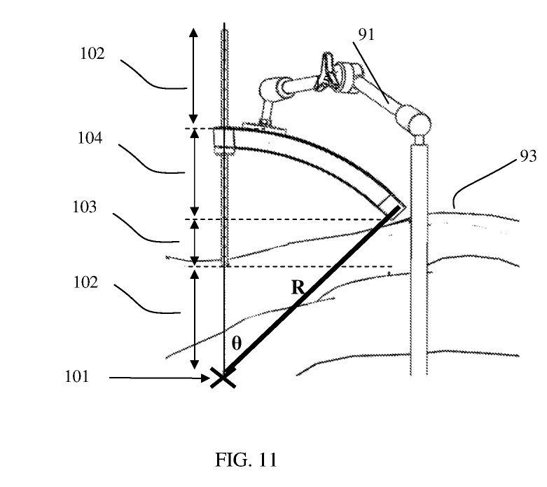

The pivot mechanism 11 enables attaching the surgical instrument guiding apparatus 100 to a supporting structure in such a way that the surgical instrument guiding apparatus is pivotable about a center of spherical rotation of the surgical instrument guiding apparatus positioned in space along the length of the longitudinal axis of the pivot mechanism. The pivot mechanism 11 comprises a pivot element 12A, a sleeve 12B and a slider assembly 13. The pivot element 12A can be a simple circular rod that can be made of stainless steel, aluminum or other materials. Top end of the pivot element 12A can be secured to a support structure (for example, support structure 91 shown in FIG. 10 and FIG. 11). The sleeve 12B is coupled to the pivot element 12A so as to rotate about the pivot axis 17 of the pivot element 12A in both directions. The rotation of the sleeve 12B relative to the pivot element 12A may not be a free rotation and restriction on relative rotation may be imparted by introducing sufficient friction. A friction hinge can be used for this purpose so that no tightening is required or it can be secured through a tightening mechanism such as a grip or a screw or any other means.

In some embodiments, the sleeve 12B can be in the form of a threaded cylinder made of stainless steel, aluminum or other rigid materials that can rotate around the pivot element 12A through a thread system or closely fitting circular surfaces. The sleeve 12B may also be in the form of a turnbuckle or a short cylinder that has both left- and right-hand threads with accompanying screws so that turning the sleeve 12B will move the whole apparatus 100 up and down relative to the patient.

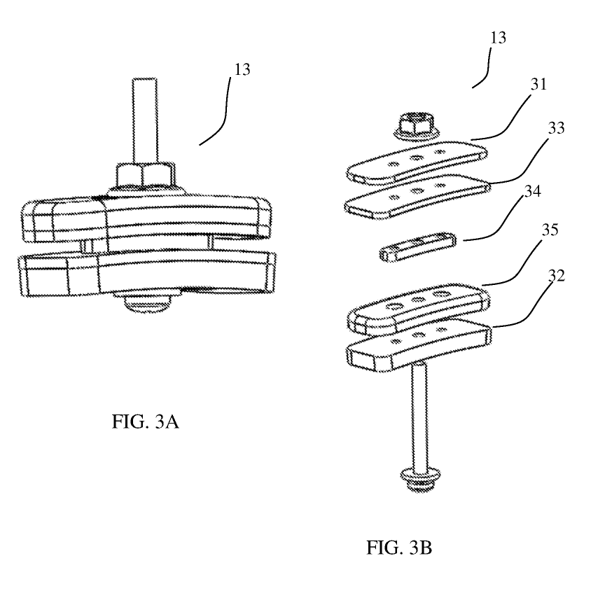

The slider assembly 13 is operatively connected to and pivots with the sleeve 12B. The slider assembly 13 is a suitably shaped plastic or aluminum component that may have a variety of possible shapes to allow the arcuate arm 14 to slide along it. In one embodiment, as illustrated in FIG. 3A and FIG. 3B, the slider assembly 13 can be an assembly of components with circular side profiles built from rigid support components 31 and 32 and low friction components 33, 34 and 35 that allow sliding of the arcuate arm 14 without play. Alternatively, components 31, 34 and 32 can be made from rigid components coated with a low friction film, such as Teflon or other low friction coefficient or self-lubricating materials.

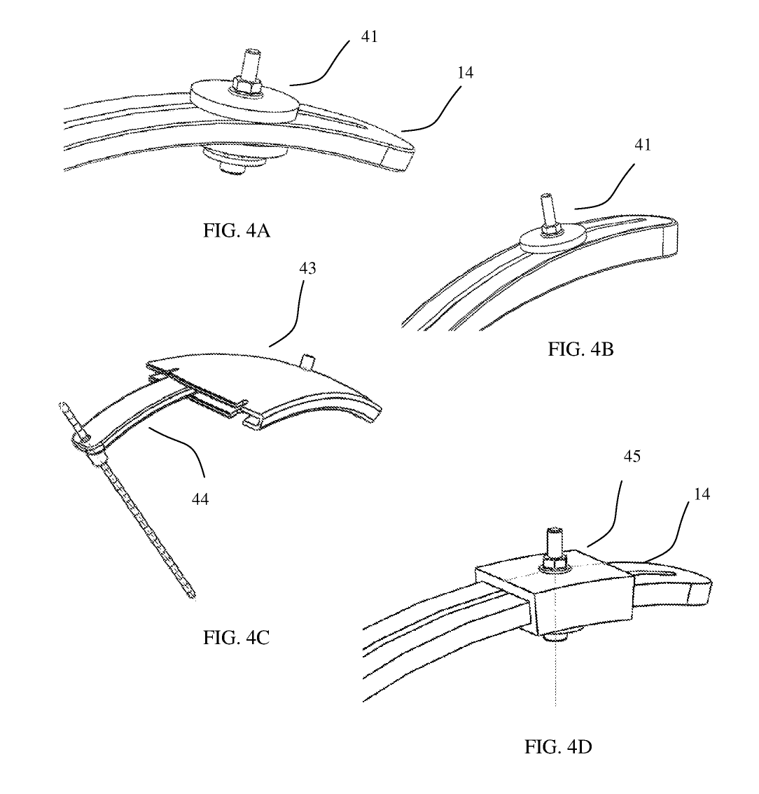

Reference to FIGS. 1 and 2, the arcuate arm 14 is mounted to the pivot mechanism 11 with the help of the slider assembly 13 which enables the arcuate arm 14 to slide/rotate along a circular path 18 centered around the center of spherical rotation 20 of the surgical instrument guiding apparatus. In accordance with the present invention, there can be various different designs of the arcuate arm 14 and slider assembly 13 which can enable the slideable engagement between the arcuate arm 14 and the slider assembly 13. FIG. 4A to FIG. 4D illustrate some of such combinations of the arcuate arm 14 and the slider assembly 13. In FIG. 4A and FIG. 4B, the slider assembly 41 comprises substantially spherical shaped disc which is rotatably and slidably received by the elongated slot disposed on the arcuate arm 14. FIG. 4C shows a closed type slider assembly 43 having a thorough cavity which allows the arcuate arm 44 to slide through it when operated. In FIG. 4D, the arcuate arm 14 is slidably enclosed by a slider assembly 45 which engages with the sides of the arcuate arm 14.

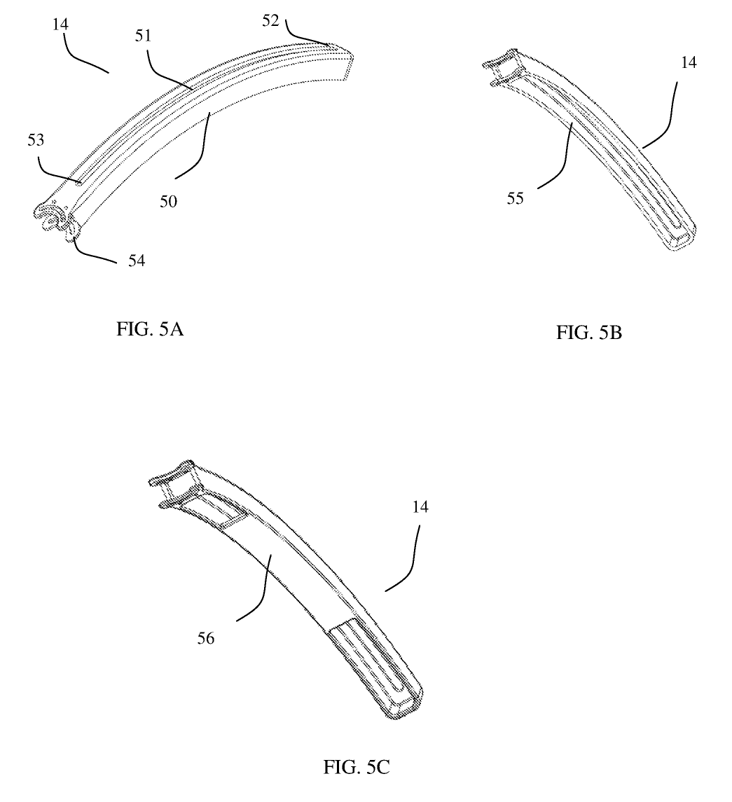

FIG. 5A to FIG. 5C show different designs of the arcuate arm 14 structure. The arcuate arm 14 comprises an arm body 50, a needle guide holder 54 disposed at one of the arm body, a channel or slot 51 longitudinally extending over at least a portion of the arm body 50 from first end 52 to second end 53. The channel 51 is designed to slidably and rotatably receive a slider assembly. The arm body 50 can have a solid body as shown in FIG. 5A or it may have a cavity 55 as shown in FIG. 5B to improve rigidity while maintaining radio-transparency. The embodiment of FIG. 5B provides a good compromise for ease of manufacturing, structural rigidity and X-ray transparency. Furthermore, the arm body can be reinforced with a variety of structural elements, such as ribs or a bridge 56 (which can be a single segment or in several segments), as shown in FIG. 5C, for increased rigidity for a selected radius and material thickness. The arm can be made of a variety of materials using a number of different production processes. The material and thicknesses of various walls of the arcuate arm are selected based on factors such as X-ray transparency, structural rigidity and integrity depending on the selected arc length and radius. For example, for the surgical instrument guiding apparatus of the present invention having an arcuate arm arc length of 300 mm and a width of 25 mm, the arcuate arm can be made of transparent surgical grade ABS with a thickness of 3 mm. However, there are a range of possible materials that would allow some freedom in design, such as carbon fiber reinforced epoxy, glass, quartz fiber reinforced composites, Lexan or Aerogel,—to list a few examples.

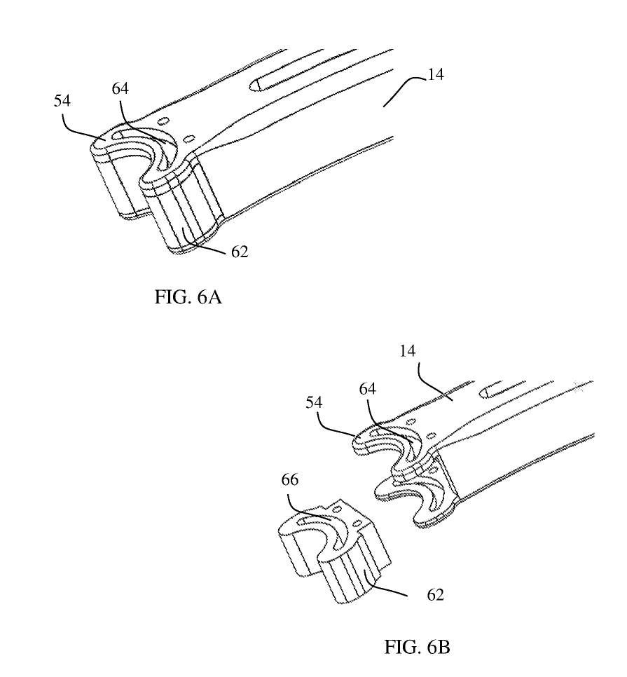

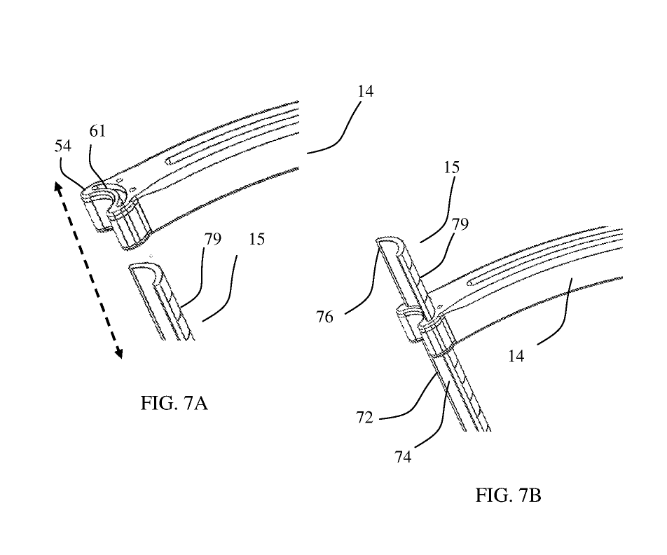

The needle guide holder 54, as shown in FIG. 6A, FIG. 6B, FIG. 7A and FIG. 7B, is configured to slidably receive the needle guide 15. In a preferred embodiment, the needle guide holder 54 can be an integral part of the arcuate arm 14 (FIG. 6A) or it can be a detachable and replaceable holder 62 releasably affixed to the arcuate arm 14 as shown in FIG. 6B. In one embodiment, the needle guide holder 54 comprises an arcuate shaped body having an arcuate shaped slot 64 extending therethrough. The needle guide holder and/or the needle guide holder slot is always designed to accommodate the profile of the needle guide 15 received by the needle guide holder. This profile may be arcuate or it may have a variety of different shapes. To introduce an optimum level of coefficient of friction, the inner surface of the needle guide holder slot can be sprayed with a low friction coefficient material or lubricant or it can alternatively be made of a plastic material like Teflon or other self-lubricating materials.

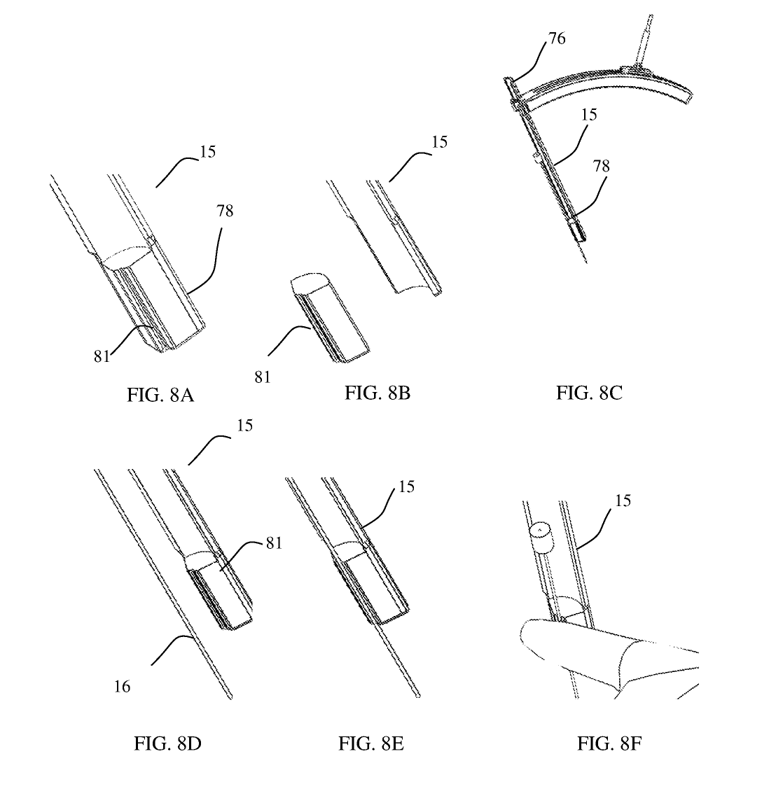

The needle guide 15, as shown in FIGS. 7A to 8F, comprises an elongate body 72 having a generally curved face 74 and is configured to support and guide a surgical instrument to move along a radial axis 19 perpendicular to the circular path 18. In a preferred embodiment, the cross-sectional profile of the elongate body 72 is made similar to that of the arcuate shaped slot 64 (shown in FIGS. 6A and 6B) when viewed in transverse cross-section along a longitudinal axis of the needle guide 15. The length of the needle guide 15 should be at least equal to the radius of the movement of the arcuate arm, i.e. the radius of the main sphere. The proximal end 76 of the elongate body 72 which comes in contact with the needle guide holder 54 is provided with a plurality of graduated grooves 79 extending parallel to the elongation of the body and being regularly spaced from each other to facilitate controlled and, optionally measured, sliding of the needle guide 15 through the needle guide holder 54. The needle guide 15 can be secured in position using friction or can be locked into position through a locking mechanism such as a screw or a spring loaded lock for more secure locking.

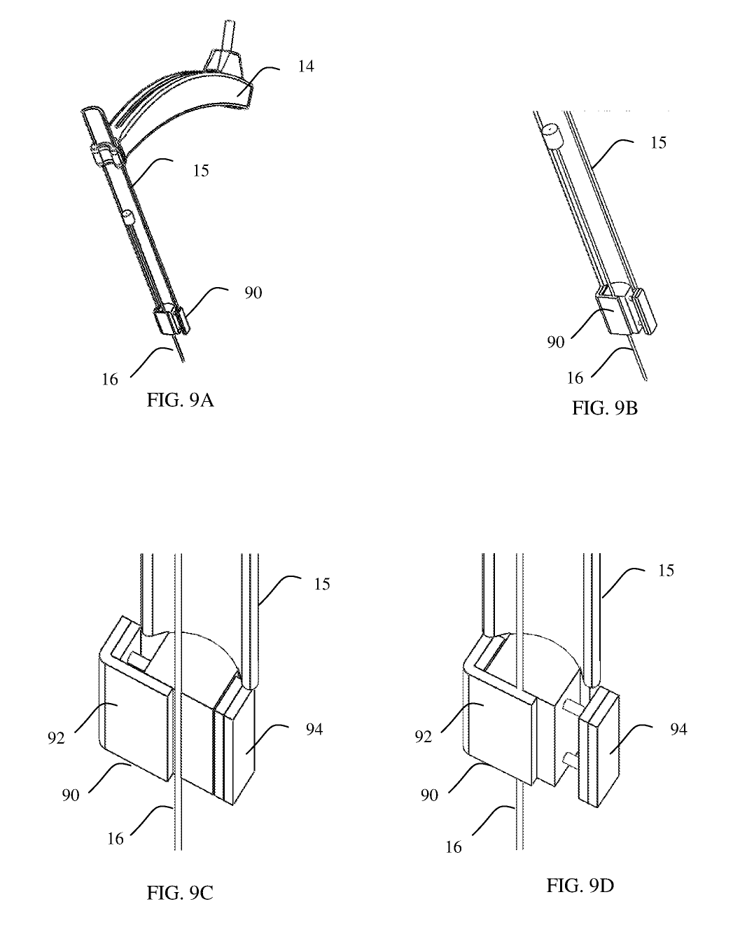

The distal end 78 of the needle guide 15 is disposed with a needle slider or needle lock. FIGS. 8A to 8F illustrate one embodiment of the needle lock 81 whereas FIGS. 9A to 9D illustrate another embodiment of the needle lock 90. The needle lock 81 or 90 is configured to slidingly support and guide a surgical instrument (e.g. a needle 16 or a catheter) to slide along a radial axis 19 that meets the pivot axis 17 of the pivot mechanism 11 at the center of spherical rotation 20 (axes 17 and 19 and center 20 are shown in FIG. 1) of the surgical instrument guiding apparatus. The needle guide 15 thus makes the needle always pass through the center of the sphere 20 in all relative positions of the components of the apparatus for a given position of the pivot mechanism 11.

In an exemplary use case, the needle 16 can be held against the needle lock 81 with a finger or two to ensure that the needle slides along the intended tract. In case of needle lock 90, the locking mechanism comprises a first part 92 and a second part 94. As shown in FIG. 9D, the first part 92, when pressed, can hold a needle or interventional component 16 tightly and/or slidably placed along the axis of the needle guide 15. The second part 94, as shown in FIG. 9C, when pressed, pushes the first part 92 to disengage the needle 16 from the needle lock 90. Again, the needle lock 81 or 90 can be an integral part of the Needle Guide 15 or a low-friction component detachably secured to it.

The thickness and radius of curvature of the needle guide's cross section depend on the selected material and the required ease of manipulation of the needle or catheter by the surgeon or the operator. For the envisaged application, if the needle guide is made of a material such as ABS, and of length of 300 mm, the guide's thickness can be in the nominal range of 2.5-4.0 mm and the radius of curvature in the nominal range of 25-35 mm Another parameter to consider is the arc length of the cross section of the needle guide body. This will affect the rigidity of the needle guide. Depending on the particular material selected, the thickness and the radius of curvature, this can have a range between 90 degrees and 180 degrees. Naturally, supporting ribs in the longitudinal direction can be used to increase stiffness for selected parameters for the needle guide.

An example of the structure needed to support the apparatus 100 in position above the patient is illustrated in FIG. 10. The supporting structure 91 may be any adjustable surgical structure such as articulated arms, sometimes known as “Swiss arms” that can be moved in a range of positions and centrally locked. It can also be any structural support commonly used in surgery to support the patient's limbs or anesthesia devices and systems that are normally secured to the rail of the operating table 92. The patient 93 in this illustration is shown in the prone position. The proposed system can work with the patient in supine or other positions as is appropriate for the surgical procedure. Alternatively, the system can be secured to the body of the patient to accommodate any motion/movement of the patient during the procedure. A commercially available height adjustment mechanism can be attached to the supporting structure to allow for fine adjustment of the spherical mechanism's height relative to the patient.

Reference to FIGS. 1, 10 and 11, be it during sliding motion of the arcuate arm 14 through the slider assembly 13 along an arc 18 or rotation of the arcuate arm 14 about the pivot axis 17, the needle 16 will always target the center of rotation 20 when moved radially inward if the longitudinal axis (radial axis) 19 of the needle 16 is maintained normal to the arcuate arm 14. Thus, an operator (e.g. a surgeon) can manipulate the apparatus 100 attached to a support structure (for example, support structure 91 shown in FIGS. 10 and 11) about a plurality of different axes relative to the support structure or to the patient as required without letting the surgical instrument (needle 16 in the present example) to go off target once the target point is set at the center of spherical rotation 20 of the apparatus 100. Only the pivot axis 17 remains fixed for the duration of the procedure. The spherical mechanism of the apparatus 100 enables the operator to insert the interventional components to move in such a direction so as to move along a radius of the sphere to pass through the center of the sphere. Since the operator has a two dimensional, real-time view of the interventional means and the target, this device will enable the operator to slide the interventional means to the desired target point.

In summary, the three degrees of freedom: rotation around pivotal axis 17, sliding along circular arc 18 and sliding along radial axis 19 allow the target to be reached by the needle or interventional component 16 from a range of locations on the patient's body.

Design and Parametric Considerations: The most important design parameter is the radius of the system sphere. The radius determines a number of aspects of the system. The overall aim of the design process is to minimize the radius of the system sphere within the constraints of range of coverage for compactness. The design of the proposed system is governed by a number of considerations. These are illustrated in FIG. 11.
1. The maximum depth 102 of the target organ 101 beneath the upper surface of the body of the patient along a line of the x-rays, designated as Dₘₐₓ.
2. The maximum elevation of the upper surface of the patient's body 93 from the point of entry to the lowermost point of the system, 103, designated as D_{w-body}.
3. The dead depth 104 due to the particular shape of the spherical guide system Depth of target variable, designated as D_{w-guide}.
4. The angular range of the device, θ

It can be readily seen that the minimum system sphere radius R (hence the radius of the Curved Arm) is given by:

R=(Dₘₐₓ+D_{w-body})/cos(θ)

The aim is generally to use the minimum possible R (to reduce device size and maximum height over patient) for a good coverage of Target depth and range angle. As an example, a system radius of 300 mm would enable coverage of a maximum organ depth of about 120 mm and yield a range angle of 50 degrees with a body surface elevation of about 70 mm in the desired range. Other combinations are naturally possible.

Upon detailed investigation of materials and their properties, it is possible to design a system with these parameters using surgical grade transparent ABS with a wall thickness of 3 mm and device outer width of 25 mm resulting in a system that weighs less than 150 g.

Although detailed for PCNL entry guidance, it is evident that the present invention can be used to reach any target in the body the location of which is known prior to the operative procedure. The apparatus can be used with the patient in the prone, supine or other positions. The apparatus can be made as re-usable, and therefore sterilizeable, or as a single-use, low cost disposable system.

What is claimed is:

1. An apparatus for guiding a surgical instrument to a target location, said apparatus comprising:
a pivot mechanism configured to be mounted to a supporting structure, said pivot mechanism is pivotable about a pivot axis passing through a center of spherical rotation of said apparatus;
an arcuate arm operatively engaged to said pivot mechanism, said arcuate arm is rotatable along a circular path centered around said center of spherical rotation; and
a needle guide mounted to said arcuate arm, said needle guide is configured to guide said surgical instrument to move perpendicular to said circular path along a radial axis;
wherein, by setting said target location at said center of spherical rotation said arcuate arm is operable in any spatial orientation for positioning said surgical instrument at a desired entry point and at a desired angle to access said target location.

2. The apparatus as in claim 1, wherein said pivot axis and said radial axis intersect at said center of spherical rotation.

3. The apparatus as in claim 1, wherein said arcuate arm is arc-shaped.

4. The apparatus as in claim 1, wherein a length of said needle guide is at least equal to a radius of said circular path.

5. The apparatus as in claim 1, wherein said needle guide is slidably mounted to said arcuate arm.

6. The apparatus as in claim 1, wherein a cross-sectional profile of an elongate body of said needle guide is similar to an arcuate shaped slot disposed on said arcuate arm.

7. The apparatus as in claim 6, wherein a plurality of graduated grooves are disposed over at least a portion of said elongate body.

8. The apparatus as in claim 1, wherein a needle lock is disposed at a distal end of said needle guide to guide said surgical instrument.

9. A method for guiding a surgical instrument to a target location using an apparatus, said method comprising:
mounting a pivot mechanism of said apparatus to a supporting structure, said pivot mechanism is configured to be pivotable about a pivot axis passing through a center of spherical rotation of said apparatus;
engaging an arcuate arm operatively to said pivot mechanism, said arcuate arm is rotatable along a circular path centered around said center of spherical rotation; and
mounting a needle guide to said arcuate arm, said needle guide being configured to guide said surgical instrument to move perpendicular to said circular path along a radial axis;
wherein, by setting said target location at said center of spherical rotation said arcuate arm is operable in any spatial orientation for positioning said surgical instrument at a desired entry point and at a desired angle to access said target location.

10. The method as in claim 9, wherein said pivot axis and said radial axis intersect at said center of spherical rotation.

11. The method as in claim 9, wherein said arcuate arm is arc-shaped.

12. The method as in claim 9, wherein a length of said needle guide is at least equal to a radius of said circular path.

13. The method as in claim 9, wherein said needle guide is slidably mounted to said arcuate arm.

14. The method as in claim 9, wherein a cross-sectional profile of an elongate body of said needle guide is similar to an arcuate shaped slot disposed on said arcuate arm.

15. The method as in claim 14, wherein a plurality of graduated grooves are disposed over at least a portion of said elongate body.

16. The method as in claim 9, wherein a needle lock is disposed at a distal end of said needle guide to guide said surgical instrument.
